(19) United States
(12) Patent Application Publication
(10) Pub. No.: US 2008/0242732 A1
(43) Pub. Date: Oct. 2, 2008

(54) Liver selective drug therapy

(76) Inventor: Howard J. Smith, Lower Templestowe (AU)

Correspondence Address: BIO INTELLECTUAL PROPERTY SERVICES (BIO IPS) LLC, 8509 KERNON CT., LORTON, VA, 22079, US

(21) Appl. No.: 11/819,279

(22) Filed: Jun. 26, 2007

Related U.S. Application Data
(63) Continuation-in-part of application No. PCT/AU00/01337, filed on Nov. 1, 2000.

(30) Foreign Application Priority Data
Nov. 3, 1999 (AU) AU PQ3855
Jan. 24, 2000 (AU) AU PQ5236
Feb. 7, 2000 (AU) AU PQ5471

(51) Int. Cl. A61K 31/135; A61P 9/12

(52) U.S. Cl. 514/652

(57) ABSTRACT

A method of pharmaceutical therapy comprising administering at least one pharmaceutical, complementary medicine, or herbal product, orally at a dose sufficient to provide a clinically effective blood level in the portal vein and less than that required to provide a clinically effective blood level in the peripheral circulation to thereby provide a dose-delivery rate having a selective clinical effect in the liver.

This application is a continuation-in-part application of international patent application PCT/AU00/01337, filed Nov. 1, 2000, and published May 10, 2001, which claims priority benefit of Australian patent applications PQ 5471, filed Feb. 7, 2000, PQ 5236, filed Jan. 24, 2000, and PQ 3855, filed Nov. 3, 1999. The disclosures of international patent application PCT/AU00/01337, and Australian patent applications PQ 5471, PQ 5236, and PQ 3855, are hereby incorporated by reference.

### DESCRIPTION OF THE INVENTION

1. Field of the Invention

The present invention relates to methods of drug treatment where the liver or portal venous circulation is the primary therapeutic target, and in particular to methods of treatment or prevention of diseases, that are selective for the liver and thereby minimize side effects. Also included are methods of the treatment of systemic diseases where the therapeutic management is directed towards a physiological or disease process acting within the liver itself.

2. Background of the Invention

The traditional methods of oral therapy for management of any disease usually require a drug to be administered by mouth to reach systemic levels of active agent within the body and circulation and to achieve the desired therapeutic effect. Since all substances absorbed from the gastrointestinal tract are then released into the portal venous circulation, they must then pass through the liver before entering the systemic circulation. The liver is generally and correctly perceived as an obstruction to the systemic bioavailability of a drug because many substances are excreted from the body through hepatic metabolism. The phenomenon of rapid uptake followed by metabolism of drugs during their first exposure to the liver is known as first-pass clearance by the liver.

This phenomenon of first-pass clearance, together with later uptake and metabolism during subsequent transits of the liver is the principal cause of a short half-life of a drug. The problem of short half-life may be addressed by 1) using loading doses of a drug to ensure that adequate systemic levels are achieved, 2) administering a drug several times a day, 3) administering the drug by a different route, for example parenterally or transdermally, or 4) developing medicines that are not taken up or metabolized by the liver, and hence have a long half-life.

### SUMMARY OF THE INVENTION

In accordance with this invention, a method of pharmaceutical therapy including orally administering at least one pharmaceutical at a dose-delivery rate sufficient to provide a clinically effective blood level in the portal vein and less than required to provide a clinically effective blood level in the peripheral circulation to thereby provide a dose-delivery rate having a selective clinical effect in the liver is provided. (Within this specification, “drug” and “pharmaceutical” may be used interchangeably.) The pharmaceutical may be administered in a slow release formulation to provide a clinically effective blood level in the portal vein, wherein the dose-delivery rate is less than required to provide a clinically effective blood level in the peripheral circulation. The pharmaceutical may be chosen from beta-blockers, statins, antioxidants and antiviral agents, or any other class of pharmaceutical where it is appropriate to concentrate or limit its therapeutic effect to the liver or portal circulation. The pharmaceutical may be administered to a patient suffering at least one disease chosen from portal hypertension, hypercholesterolemia, autoimmune disease, hepatitis, including viral hepatitis, and hepatic hypoxia.

The present invention also provides a method of treating a patient suffering portal hypertension by orally administering a slow release formulation of at least one beta-blocker to provide a dose-delivery rate sufficient to provide beta-blockade in the liver and portal system, but less than required to provide a blood level in the peripheral circulation to have an effect of inhibition of heart rate. The beta-blocker may be propranolol, and the beta-blocker may be administered as a slow-release formulation at a dose equivalent to from 10 to 25 mg per day of propranolol.

Other aspects of the invention provide a method of treating a patient suffering from hypercholesterolemia by orally administering a slow-release formulation of at least one statin compound. In some embodiments, the statin compound has the formula:

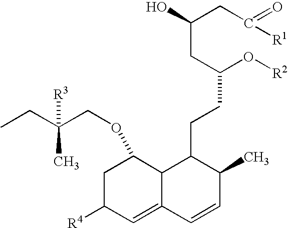

wherein:
R¹ is OR⁵, wherein R⁵ is a counter ion such as sodium,
R³ is a hydrogen or methyl,
R⁴ is chosen from hydrogen, hydroxy and methyl,
R² is hydrogen, or R¹ and R² may together form a bond to provide a lactone;
wherein the slow-release formulation provides a dose-delivery rate sufficient to provide a cholesterol lowering effect in the liver and less than required to provide inhibition of systemic synthesis of ubiquinone. The compound may be chosen from simvastatin, pravastatin, mevastatin, lovastatin, fluvastatin, cerivastatin, rosuvastatin, atorvastatin, and derivatives thereof. In some embodiments, the compound is administered in a slow-release formulation providing a dose equivalent to from 1 to 40 mg per day of simvastatin.

The present invention also provides a method of treating a patient suffering from autoimmune hepatitis by administering to the patient at least one steroid effective in treating hepatitis, wherein the steroid is administered orally in a blood level to produce systemic effects. In some embodiments, the steroid is prednisone or another corticosteroid.

In other embodiments, a method of treating a patient suffering from hepatic hypoxia is provided, comprising orally administering to the patient at least one antioxidant in a slow-release formulation at a dose-delivery rate sufficient to provide an effective blood level in the portal system, but less than that capable of producing blood levels in the peripheral circulation sufficient to produce clinical or adverse effects.

In still other embodiments, the invention provides a method of treating a patient suffering from a form of liver disease other than portal hypertension, autoimmune hepatitis, or hepatic hypoxia, comprising administering to the patient a slow release formulation of at lease one drug sufficient to achieve effective blood levels in the liver and portal venous system, but less than that required to produce clinical or adverse effects elsewhere in the body.

In still other embodiments, the present invention provides a method of treating a patient suffering from a form of liver disease other than portal hypertension, hypercholesterolemia, hepatitis, viral hepatitis, or hepatic hypoxia, comprising administering to the patient a slow release of at least one complementary medicine or herbal product sufficient to achieve effective blood levels in the liver and portal venous system and less than that required to produce clinical or adverse effects elsewhere in the body.

Additional aspects of the invention will be set forth in part in the description which follows, and in part will follow from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE EMBODIMENTS

In accordance with the present invention, we provide a method of pharmaceutical therapy comprising administering a pharmaceutical orally at a dose sufficient to provide a clinically effective blood level in the portal vein and liver, but less than that required to provide a clinically effective blood level in the peripheral circulation. The method thereby provides a dose-delivery rate with a clinically selective effect in the liver.

Diseases of the liver and portal circulation to which this invention applies include portal hypertension resulting from cirrhosis of the liver, hypercholesterolemia, viral hepatitis of any form including hepatitis A, B, C, D, E, G, and other viral infections, autoimmune hepatitis, hepatic hypoxic conditions resulting from primary disease of the liver or secondary to extrahepatic diseases, and any other condition where the liver itself is the primary therapeutic target and it is desirable to concentrate a therapeutic agent within the liver.

The present invention provides a method of administering a drug with an intrinsic short half-life, at a low dose, and in a slow-release formulation. In this way, clinically effective concentrations of a drug will be achieved in the portal circulation and within the liver itself. However, clinically effective blood levels will not be achieved in the peripheral or systemic circulation because 1) a significant portion of the drug is removed by the liver during first-pass, and 2) the relatively large volume of the systemic circulation compared with the smaller volume portal circulation creates a dilution effect. It is therefore an underlying principle of this invention that the short half-life of a drug becomes a strength rather than a weakness, and can be employed to achieve relative selectivity of a therapeutic effect.

The principles of liver-selective delivery of drugs apply to any condition where the liver or portal venous circulation is the primary target for drug treatment. Many diseases are presently treated with systemic doses of established drugs, or are intended to be treated with novel classes of drugs presently in development. It is a key principle of this invention that the use of low-dose, slow-release formulations of these drugs will achieve the desired therapeutic effect in a manner similar to, or more effective than present treatment, but with a much lower rate of systemic side effects. Thus, the use of liver-selective delivery of drugs for treatment of liver disease can expect a greater tolerance, acceptance and compliance by patients.

The methods of the invention may involve the oral administration of a pharmaceutical at a dose-delivery rate sufficient to provide a clinically effective blood level in the portal system, but less than that required to provide a clinically effective blood level in the peripheral circulation. The dose-delivery rate is typically achieved by a slow release formulation.

The principle of achieving liver-selectivity by use of a slow release formulation also applies to the use of a slow infusion of a medicine into the gastro-intestinal tract through a naso-gastric tube or other artificial access. While such a route of administration will usually be impracticable for chronic treatment, the use of this technique in situations of acute medical care may ensure delivery of a therapeutic agent to the body, and at the same time minimize systemic side effects.

The principle of liver-selective delivery of drugs can be described mathematically in the following way.

Consider a drug administered by mouth as a slow-release formulation to achieve steady state release into the bowel with uptake into the portal venous circulation. The drug is then partly metabolized by the liver.

Let the volume of blood passing through the portal circulation in unit time=Vp liters.
Let the total volume of the systemic circulation=V_{S} liters.
Let the concentration of drug in the portal vein=C_{P} mg/liter.
Let the concentration of drug in the systemic circulation=C_{S} mg/liter.
Let the drug absorbed from the GI tract in unit time=D_{A} mg.
Let the drug metabolized by the liver in unit time=D_{M} mg
Let the drug not metabolized by the liver in unit time=D_{A}−D_{M} mg=D_{NM} mg
Let the metabolic clearance=M
This must range from 0 (no clearance) to 1.0 (total clearance).
Then C_{P} is determined by the amount of drug absorbed into the finite V_{P} plus the concentration of the drug recirculated.

C_{P}=D_{A}/V_{P}+C_{S}

i.e., D_{A}=V_{P}(C_{P}−C_{S}) equation. 1

The amount of the drug metabolized is a function of clearance rate, portal venous concentration, and portal volume per unit time.

D_{M}=M×C_{P}×V_{P} equation. 2

The systemic concentration of drug is determined by the volume of the systemic circulation and the amount of drug not metabolized.

C_{S}=D_{NM}/V_{S}

i.e., D_{NM}=C_{S}×V_{S} equation. 3

By definition,

D_{A}=D_{M}+D_{NM}

Substituting equations 1, 2, and 3,

V_{P}(C_{P}−C_{S})=M×C_{P}×V_{P}+C_{S}×V_{S}

and

C_{P}[V_{P}(1−M)]=C_{S}(V_{S}+V_{P})

such that

C_{P}/C_{S}=(V_{S}+V_{P})/V_{P}(1−M)

This relationship may be interpreted in the following way.
1. When there is no metabolic clearance of a drug by the liver, (M=0), the concentration gradient between portal and systemic vessels during steady state release of a drug from a slow-release formulation is a function of their relative volumes of the two circulations.

C_{P}/C_{S}=(V_{S}+V_{P})/V_{P}

2. With total hepatic clearance, M=1, and C_{P}/C_{S} tends towards infinity.
3. If the rate of metabolism by the liver saturates, M will decline at higher dose levels. Therefore liver selectivity will be greater at lower dose levels, and be maximal when there is no effective saturation of metabolism.
4. Portal venous flow does vary. Therefore C_{P}/C_{S} will be higher under low-flow conditions, for example in cirrhosis, but be low in high-flow situations such as when there is an abnormal shunting of blood perhaps through fistulae.

### Treatment of Portal Hypertension

A specific example of liver-selective delivery is liver-selective beta-blockade for the treatment of portal venous hypertension. The present invention therefore relates to a method of treatment of portal hypertension and prevention of variceal bleeding.

Portal hypertension is a common complication of cirrhosis of the liver and is defined by the elevation of venous pressure in the portal vein to levels>30 cm saline.

The portal vein is the final common conduit for blood draining the major part of the gastrointestinal tract including stomach, and both the small and large bowel, and passing to the liver. Because the portal vein lacks valves, any obstruction to the flow of blood within the liver, within the portal vein itself, or by elevation of pressure in the inferior vena cava, causes elevation of the pressure in the portal vein and its tributaries. In practice, the most common cause of portal hypertension is cirrhosis of the liver, of which the most common cause is end-stage alcoholic liver disease. In the USA, clinically significant portal hypertension is present in more than 60% of patients with cirrhosis.

The symptoms of portal hypertension are usually superimposed on the symptoms of the underlying liver disease and impaired liver function. They include the physical effects of raised portal vein pressure—hemorrhage from gastro-esophageal varices (variceal bleeding), splenomegaly with hypersplenism, and ascites, which is fluid leak into the peritoneal space. Acute hemorrhage into the bowel from bleeding varices is the most serious complication, and may produce acute shock and death. It is therefore a life-threatening emergency. Milder cases of hemorrhage may present as melena, which is usually interpreted as a warning of potential massive hemorrhage.

The treatment of variceal bleeding includes conventional methods of blood and fluid replacement to restore blood volume and pressure. In addition, local treatment with balloon tamponade, sclerosis of varices and selected vasoconstrictors may be employed.

Prevention of variceal bleeding utilizes techniques that can lower portal venous pressure and thereby reduce the chance of rupture. Several surgical techniques have been developed but these are by their nature invasive. An alternative method has been to administer beta-adrenergic antagonists (beta-blockers), particularly propranolol. Beta-blockers inhibit the action of the beta-adrenergic effect of adrenaline throughout the body, including the constrictor effect of adrenaline on the portal vein. Therefore, they act to lower portal venous pressure, and have been shown to prevent a first variceal bleed and subsequent episodes after an initial bleed.

The use of beta-blockers, such as propranolol, in patients with portal hypertension and advanced liver disease has up until now not been widely accepted because the systemic effects of the drug are cardiac-related, with potential adverse effects in these patients. Beta-blockers slow the heart, lower blood pressure, and may mask the early signs of shock in a patient who is bleeding internally. Beta-blockers frequently cause both fatigue and lethargy, which are common symptoms in patients with liver disease. Since propranolol is also metabolized by the liver, the inability of an impaired liver to clear the drug from the circulation when the drug is given in normal systemic doses may cause plasma levels to rise thereby exacerbating cardiac symptoms, and in severe cases precipitate encephalopathy.

Therefore, while current medical textbooks note the potential of propranolol to lower portal venous pressure and reduce variceal hemorrhage, the prescribing information for propranolol in most countries specifically warns against the use of the drug in patients with decompensated cirrhosis, noting that encephalopathy may develop and symptoms of hemorrhage may be masked.

In this aspect of the invention, a method of treatment of portal hypertension including the administration of propranolol in a form selective for the liver that will reduce portal venous pressure with minimal risk of adverse systemic effects is provided. The method involves use of a slow-release formulation of a low dose of a beta-blocker such as propranolol, being a drug that is metabolized by the liver with relatively high first pass clearance. In this way, clinically effective blood levels of the drug will be achieved in blood reaching the liver and the portal circulation, but not the peripheral blood circulation.

In the treatment of portal hypertension, the primary target of the beta-blocker drug is the portal circulation, that is, a circulatory level before the drug is cleared from the circulation by the liver. Therefore, the requirement is for effective plasma concentrations of the drug in blood that has not yet passed through the liver.

This is in contrast to the treatment of cardiac conditions where a drug must clear first-pass metabolism by the liver and then disperse throughout the much larger systemic blood volume. Therefore, when a drug is given as a low-dose sustained release formulation, effective plasma concentrations of the drug will be achieved in the portal circulation at lower daily doses than are required to achieve systemic effects. Two other features of cirrhosis with portal hypertension also act to reduce the rate of drug metabolism by the liver. Impaired liver function itself reduces drug clearance and venous obstruction reduces portal blood flow. This means that the daily dose of slow-release of formulation of propranolol required to achieve clinically useful blood levels in the portal circulation may be as low as one tenth to one twentieth of those required to achieve systemic effects, for example, the doses used to treat cardiac disease. Thus, while the dose of propranolol used in systemic doses to treat portal hypertension is in the range of 80-160 mg or more per day, the dose used as a liver-selective formulation will be in the range 10-25 mg per day. The daily dose will be least in those patients with the most severe cirrhosis of the liver because very slow portal venous blood flow is a feature of this condition. In any patient, the optimum dose should be the highest dose that does not produce evidence of systemic beta-blockade as evidenced by inhibition of tachycardia.

Compounds for treatment of portal hypertension include beta- adrenergic antagonists (beta-blockers) that are non-selective (having both beta-1 and beta-2 properties), and are metabolized by the liver. This includes almost all lipophilic beta-blockers including propranolol, nadolol, oxprenolol, and other compounds. These compounds have a short half-life, where the half-life is a function of metabolism by the liver. This is contrary to the discipline of drug development, which has, where possible, selected agents with longer half-lives to allow once-a-day administration. In the present invention, the slow-release formulation enables a continuous low dose to be delivered to the liver and the portal circulation, and achieve therapeutic blood levels, without reaching clinically significant levels in the peripheral circulation.

### Treatment of Hypercholesterolemia

The present invention also relates to a method of treatment of hypercholesterolemia, and in particular to a method of treatment of hypercholesterolemia using HMG-CoA reductase inhibitors, such as the statin class of drugs, being compositions containing HMG-CoA reductase inhibitors.

Atherosclerosis and its various clinical presentations as coronary artery disease, cerebrovascular disease, peripheral vascular disease and other conditions, is a major cause of death in western countries. Hypercholesterolemia is a primary risk factor for death from these conditions. HMG-COA reductase (3-hydroxy-3-methyl-glutaryl-coenzyme A) catalyzes the rate-determining step in cholesterol biosynthesis (conversion of HMG-COA to mevalonate), and inhibitors of HMG-COA reductase have proved to be most effective in reducing the plasma levels of cholesterol in patients with both hypercholesterolemia and normocholesterolemia. For example, simvastatin in clinical trials reduced cholesterol and LDL cholesterol by 25% and 35% respectively. Simvastatin was reported in trials to reduce the risk of a major coronary event by 34%.

The statins have been effectively used in treating individuals with high cholesterol for many years. However the treatment of patients with inhibitors of HMG-COA reductase, such as the statins, is accompanied by adverse side effects which cause discomfort and may necessitate discontinuation of medication. As HMG-CoA reductase inhibitors are often used as a long term means for prevention of heart disease in patients who may be otherwise healthy, there is a need for a method of treatment of hypercholesterolemia without the associated adverse effects of HMG-COA reductase inhibitors.

Adverse effects known to be associated with the use of HMG-COA reductase inhibitors include muscle cramps, myalgia, increased risk of myopathy, transient elevation of creatine phosphokinase levels from skeletal muscle, and even rhabdomyolysis. The risk of these side effects is further increased when some other lipid lowering drugs, for example, gemfibrizol, are co-prescribed.

The use of HMG-CoA reductase inhibitors has also been reported to aggravate cardiac function and uncommonly, to worsen cardiac failure. These adverse effects in both skeletal muscle and the heart, are not common, but appear to have a common pathway related to inhibition of the synthesis of ubiquinone.

HMG-COA reductase is a key enzyme in the synthesis of ubiquinone (also known as coenzyme Q10), because this substance is also synthesized from mevalonate. Therefore, HMG-COA reductase inhibitors cause depletion of coenzyme Q10. The role of HMG-COA reductase in synthesis of ubiquinone and cholesterol may be schematically shown as follows:

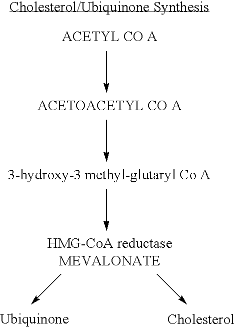

Coenzyme Q10 is a key redox coenzyme of the respiratory chain responsible for energy production within mitochondria throughout the body. These processes have been termed “bioenergetics”. Depletion of Coenzyme Q10 in skeletal and cardiac muscle has been linked to the development of both skeletal myopathy and cardiac myopathy, to the development of fatigue, and has been proposed as the mechanism of action of statin-induced muscle disease. Since fatigue is a widely reported symptom in patients with cardiovascular disease, many of whom are taking HMG-CoA reductase inhibitors for the treatment of hypercholesterolemia, it is likely that a contribution to the cause of fatigue by these drugs has not been appreciated and is therefore under-diagnosed.

U.S. Pat. No. 5,316,765 describes a method and composition for reducing the side effects of HMG-COA reductase inhibitors, which involves concurrent administration of coenzyme Q10 in an attempt to offset the clinical effects of inhibiting formation of coenzyme Q10.

Reports published in the scientific literature attest to the use in select patients of dietary ubiquinone to reverse clinically significant adverse effects of HMG-COA reductase inhibitors in skeletal muscle or presenting as cardiac dysfunction.

In this aspect of the invention, a method of treating hypercholesterolemia is provided, comprising administering an HMG-CoA reductase inhibitor in a manner that is selective for the liver, and that will reduce hypercholesterolemia without systemic depression of Coenzyme Q10 and its sequelae of muscle disease and other conditions, including those of the heart. This method involves use of a slow-release formulation of a low dose of HMG-CoA reductase inhibitor that is itself metabolized by the liver. In this way, clinically effective blood levels of the HMG-CoA reductase inhibitor will be achieved in blood reaching the liver through the portal venous system, but not in the peripheral blood circulation.

As 90% of cholesterol synthesis within the body occurs in the liver, but ubiquinone synthesis is a systemic cell process, this method of the invention provides effective cholesterol control without the same risk of side effects associated with conventional treatments.

Acceptable statin compounds for use in this invention include, but are not limited to, cerivastatin, rosuvastatin, simvastatin, lovastatin, pravastatin, mevastatin, fluvastatin, atorvastatin, and derivatives thereof. The invention may, however, be applied to any lipid-lowering agent that also depresses levels of ubiquinone (coenzyme QIO). Other examples of acceptable compounds for use in accordance with this invention include fibrates, such as gemfibrizol. Some acceptable compounds will be absorbed from all or almost all of the small bowel, and have a short half-life on account of metabolism by the liver. Others may have a longer half-life. Some compounds may be lipophilic, whereas others may be hydrophilic. Additionally, compounds may have hydrophilic or lipophilic forms.

Some statin type HMG-CoA reductase inhibitors will have the formula:

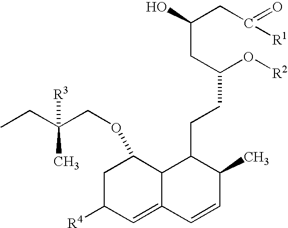

wherein:
R¹ is OR⁵, wherein R⁵ is a counter ion such as sodium,
R³ is hydrogen or methyl,
R⁴ is chosen from hydrogen, hydroxy and methyl,
R² is hydrogen, or R¹ and R² may together form a bond to provide a lactone.

In a further aspect, the invention provides for the use of an HMG-COA reductase inhibitor formulated as a slow-release pharmaceutical for treatment or prophylaxis of hypercholesterolemia.

Formulations that release the HMG-COA reductase inhibitor slowly over a time period of from about 6 to about 24 hours following administration (permitting once a day administration by mouth) will effectively control plasma cholesterol without the need to expose the peripheral circulation to active levels of the drug. The release characteristics of the slow-release formulation will provide a daily dosage of the HMG-COA reductase inhibitor at less than the dose of the drug when used in full clinical or systemic doses as a conventional formulation.

Some differences between the kinetics of HMG-CoA reductase inhibitors as a class and the beta-adrenergic antagonist propranolol need to be noted. In contrast to propranolol, simvastatin is known to have very high first pass clearance by the liver—up to 92%. This means that in contrast to propranolol, simvastatin is inherently liver-selective without the need for special formulation.

However, the exposure to the rest of the body of the 8% of drug that is not cleared by the liver appears to be sufficient to produce adverse events in some people. It is a claim of this invention that presentation of HMG-CoA reductase inhibitors as slow-release and low-dose formulations will reduce or permit avoidance of all adverse events associated with systemic depletion of ubiquinone. At the same time, delivery of an HMG-COA reductase inhibitor as a slow-release formulation can lower plasma cholesterol in a manner similar to or greater than systemic doses in conventional formulations.

In the case of simvastatin, for which the labeled dose is 5-80 mg per day, a liver selective formulation presented as a slow-release formulation will likely have a lower total dose. The final doses desired will need to be established in clinical trials, but may be in the range of 1-20 mg per day. Generally, a statin dose according to the present invention will range from about 1 mg to about 40 mg, or from about 1 mg to about 20 mg, or from about 1 mg to about 10 mg.

Generally, HMG-CoA reductase inhibitors for use in the invention are those with a short half-life, where the short half-life is a function of metabolism by the liver. This is contrary to the discipline of drug development, which has, where possible, selected agents with longer half-lives to allow a once a day administration. In the present invention, the slow-release formulation enables a continuous low dose to be delivered to the liver and achieve therapeutic levels within the liver, without reaching clinically significant blood levels in the peripheral circulation. In the present invention, formulations are designed to achieve a first-pass clearance that is greater than that of conventional immediate release formulations.

In accordance with the invention, it may also be desirable to use hydrophobic statins, or hydrophobic forms of statins, as defined by their aqueous solubility at room temperature. For example, statins having an aqueous solubility at room temperature of less than 5 grams per liter, such as fluvastatin, lovastatin, simvastatin, and atorvastatin, may be used. Also acceptable are statins having an aqueous solubility at room temperature of less than 1 gram per liter, such as lovastatin, simvastatin, and atorvastatin. With regard to simvastatin and lovastatin, the lactone forms may also be used.

### Autoimmune Hepatitis

Autoimmune hepatitis is a rare disease that benefits from chronic treatment with systemic steroids. The use of a liver-selective steroid as a low-dose, slow-release formulation is an easily understood example of the use of this invention, because the systemic effects of the chronic use of steroids are well known. These include suppression of the adrenal gland, osteoporosis, susceptibility to infection, weight gain, fluid retention, and other effects.

It is a further aspect of this invention that when used in low dose as a sustained-release formulation to achieve liver-selectivity, steroids such as prednisone may be used to treat autoimmune hepatitis without risk, or with less risk of unwanted systemic side effects.

### Viral Hepatitis

All varieties of viral hepatitis, (Hepatitis A, B, C, D, E, F, G, and others) are systemic diseases, but their principal site of activity and the principal site of viral replication is in the liver. Therefore, it is desirable to concentrate a viracidal drug within the liver to enhance its efficacy. Furthermore the required cellular effects of these drugs, their frequent need in patients with impaired immune and hemopoietic systems, and other systemic effects support the desirability of liver-selective therapy.

It is a further aspect of this invention that when used in low dose as a sustained-release formulation to achieve liver-selectivity, orally administered antiviral agents from a wide range of chemical class may be used to treat viral hepatitis with less risk of unwanted systemic side effects. Examples of oral antiviral drugs used in the management of viral hepatitis include ribavirin and related molecules.

### Hepatic Hypoxia

Ninety to ninety-five percent of the blood flow to the liver is venous flow carrying less than arterial levels of oxygen. While the liver is very capable of operating at relatively low oxygen levels, any condition that reduces venous perfusion is known to reduce intrahepatic oxygen levels to hypoxic levels and thereby reduce liver function over and beyond the depressant effect of the of the liver (in which portal venous flow is impeded by fibrosis and tissue damage), all forms of viral hepatitis where flow is impeded by swelling of the inflamed hepatocytes, other forms of hepatitis including alcoholic hepatitis, and congestion of the liver caused by cardiac failure and caval obstruction.

Hypoxia of any tissue in any organ causes elevation of intracellular reducing compounds, such as NADPH₂, which then act to contribute to the production of free radicals. Free radicals, and in particular the hydroxy free-radical, attack phospholipid within cell membranes converting small amounts to lysophospholipid. This has the effect of increasing the permeability of the membranes allowing entry of calcium ions and other substances. The membrane damage is followed by a cascade of cellular dysfunction presenting as organ dysfunction or cell death. In the case of the heart or brain, antioxidants that act to absorb free radicals can delay the hypoxic damage including infarction, but their effect is very transitory on account of the severity of the oxygen deficit that is sufficient to cause cell death. By contrast, disease processes within the liver create moderate rather than fatal hypoxia that may last for many months albeit with diminished function of the liver.

It is a further aspect of this invention that when used in low dose as a sustained-release formulation to achieve liver-selectivity, orally administered antioxidants chosen from a wide range of chemical classes may be used to treat diseases of the liver characterized by hypoxia. Administered in this way, a therapeutic effect may be achieved with no or minimal risk of systemic side effects.

It is a further aspect of this invention that when used in low dose as a sustained-release formulation to achieve liver-selectivity, orally administered antioxidants chosen from a wide range of chemical classes may be used to treat diseases of the liver characterised by hypoxia. Administered in this way, a therapeutic effect may be achieved with no or minimal risk of systemic side effects.

Examples of antioxidants that may be reformulated for liver-selective delivery include N-acetylcysteine, S-adenosyl-L-methionine, silymarin, vitamin E, polyenylphosphatidylcholine, and L-diltiazem. In contrast to D-diltiazem, which is the well-known calcium blocker drug (calcium antagonist), L-diltiazem retains antioxidant activity without the vasodilator properties of the D-enantiomer.

### Other Conditions

It is a further aspect of this invention that it applies to any other condition where the liver itself is the primary therapeutic target and it is desirable to concentrate a therapeutic agent within the liver.

### Complementary Medicines

Modern pharmacotherapy is progressively using herbal and traditional medicines to complement the use of prescription medicines. It is a further aspect of this invention that when used as a low-dose, sustained-release formulation, any orally-administered herbal or complementary medicine product chosen because of its known or perceived ability to treat liver disease, will act as a liver-selective treatment. This idea is based on the principle that the active agent or agents of a herbal or complementary product must be absorbed into the body and pass through the portal circulation and liver in the same way as any other therapeutic agent. Herbal or complementary products used to treat liver disease include milk thistle extracts in which the active agent is silymarin.

### Formulation for Slow-Release

There are many techniques to effect slow release of an active pharmaceutical agent from an orally-administered formulation. The present invention contemplates formulating a low dose of a drug with a short half-life as a slow-release formulation to produce liver selectivity, and it is intended to cover any method of slow-release formulation. These methods may include techniques designed to delay the disintegration of a capsule, tablet, or other vehicle, techniques designed to delay the solubility of a capsule, tablet or other vehicle, and techniques in which an active agent may be bound to a polymer or other large molecule such that absorption can not take place until the substance has been released from the polymer or other large molecule. The means of achieving these different methods of slow release are varied and include well known older methods, such as layers of shellac coating, and more modern techniques using synthetic and cellulose polymers.

The dosage forms according to the present invention may be controlled-release dosage forms. The mechanism of release of these dosage forms can be controlled by diffusion and/or erosion. In some embodiments, the formulation comprises polymer-coated multiparticulates, polymer-coated tablets or minitablets,

A slow release formulation may be designed to release an active agent over a period of about 6 to about 24 hours following administration, thereby permitting once-a-day administration. In some embodiments, formulations releasing a drug over extended periods of time may have more than one timed-release component to effect time coverage.

The invention will now be described with reference to the following example. It is to be understood that the example is provided by way of illustration of the invention and is in no way limiting of the scope of the invention.

### EXAMPLE

### Exemplification of the Kinetic Principle of Liver-Selective Therapy

Experiments were undertaken in four dogs under general anesthesia induced with halothane and then maintained with ketamine and xylazine. Cardiovascular status was monitored by measurement of heart rate and blood pressure and by measurement of arterial blood gases. Ventilation was assisted to maintain blood gases within physiological limits. A catheter was placed in the femoral artery to permit sampling of arterial blood. After laparotomy, a catheter was placed in a mesenteric vein and advanced to the portal vein to permit sampling of portal venous blood samples.

Propranolol was administered by mouth on the evening before, and then again one hour before the study began at a dose of 40 mg in granules taken from a 160 mg slow release formulation of propranolol (Cardinol; Pacific Pharmaceuticals New Zealand). Paired blood samples were then taken from systemic artery and femoral vein at 0, ½, 1, 1½, and 2 hour points, measured from the time of placement of the portal vein catheter, for measurement of the blood concentration of propranolol. The animals were sacrificed at the end of the experiment.

Results are displayed in the Table. The concentration in systemic blood was generally below the level of detection (<5 μg /ml).

| | Kinetic | Studies | | | |
|---|---|---|---|---|---|
| | Propranolol | Concentration | ug/ml | | |
| | Dog 1 | Dog 2 | Dog 3 | Dog 4 | Mean |
| Portal Vein | | | | | |
| Baseline | <5 | 28.8 | 21 | 36 | |
| 30 min | 11.5 | 11.8 | 13 | 2 | |
| 60 min | 5.8 | 10.9 | 10 | 6 | |
| 90 min | 22 | 14.4 | 10 | 5 | |
| 120 min | <5 | | 13 | 4 | |
| Systemic | | | | | |
| Baseline | <5 | <5 | 3 | <2 | |
| 30 min | <5 | <5 | 2 | <2 | |
| 60 min | 6.2 | <5 | 2 | <2 | |
| 90 min | 5.8 | <5 | 2 | <2 | |
| 120 min | <5 | | 2 | <2 | |

In this small series in dogs, the data indicate concentration gradients between portal and systemic vessels that provide liver selective therapy.

It is to be understood that the invention described herein above is susceptible to variations, modifications, and/or additions other than those specifically described and that the invention includes all such variations, modifications and/or additions which fall within the spirit and scope of the above description.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1.-27. (canceled)

28. A method of treatment of a patient suffering portal hypertension comprising administering orally to the patient a low dose, slow release formulation of a beta-blocker to provide dose-delivery rate sufficient to provide beta-blockade in the liver and portal system and less than required to provide a blood level in the peripheral circulation to have an effect of inhibition of heart rate.

29. The method of treatment of claim 28 wherein the beta-blocker is a lipophilic beta-blocker.

30. The method of claim 29 wherein the lipophilic beta-blocker is selected from the group consisting of propranolol, nadolol, and oxprenolol.

31. A method according to claim 30 wherein the beta-blocker is propranolol.

32. A method according to claim 28 wherein the beta-blocker is administered as a slow-release formulation at a dose equivalent to from 10 to 25 mg per day.

33. The method of claim 29 wherein the beta-blocker is administered as a slow release formation at a dose equivalent to from 10 to 25 mg per day.

34. The method according to claim 30 wherein the beta-blocker is administered as a slow-release formulation at a dose equivalent to from 10 to 25 mg per day.

35. The method according to claim 31 wherein the beta-blocker is administered as a slow-release formulation at a dose equivalent to from 10 to 25 mg per day of propranolol.
